(19) United States
(12) Patent Application Publication
(10) Pub. No.: US 2013/0245259 A1
(43) Pub. Date: Sep. 19, 2013

(54) PROCESS FOR THE PREPARATION OF BOSENTAN MONOHYDRATE

(71) Applicants: Amala Kishan Kompella, Hyderabad (IN); Srinivasu Kasa, Hyderabad (IN); Veera Swamy Balina, Hyderabad (IN); Subhash Kusumba, Hyderabad (IN); Kali Satya Bhujanga Rao Adibhatla, Hyderabad (IN); Venkaiah Chowdary Nannapaneni, Hyderabad (IN)

(72) Inventors: Amala Kishan Kompella, Hyderabad (IN); Srinivasu Kasa, Hyderabad (IN); Veera Swamy Balina, Hyderabad (IN); Subhash Kusumba, Hyderabad (IN); Kali Satya Bhujanga Rao Adibhatla, Hyderabad (IN); Venkaiah Chowdary Nannapaneni, Hyderabad (IN)

(73) Assignee: Natco Pharma Limited, Hyderabad (IN)

(21) Appl. No.: 13/422,491

(22) Filed: Mar. 16, 2012

(51) Int. Cl. C07D 403/04

(52) U.S. Cl. 544/296

(57) ABSTRACT

The invention relates to improved processes for the preparation of bosentan monohydrate which provide higher yield and purity.

### FIELD OF THE INVENTION

The present invention is concerned with a process for the preparation of bosentan monohydrate.

### BACKGROUND OF THE INVENTION

Bosentan is an endothelin receptor antagonist, belonging to a class of highly substituted pyrimidine derivatives. It is designated chemically as 4-tert-butyl-N-[6-(2-hydroxyethoxy)-5-(2-methoxyphenoxy)-2-(pyrimidin-2-yl)pyrimidin-4-yl]benzene-1-sulfonamide, and is generally used in the form of the monohydrate which has the following structure:

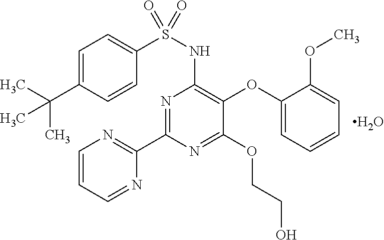

The preparation of 4-tert-butyl-N-[6-(2-hydroxyethoxy)-5-(2-methoxyphenoxy)-2-(pyrimidin-2-yl)pyrimidin-4-yl]benzene-1-sulfonamide monohydrate of formula (I) and the use thereof especially as an antihypertensive agent is described in U.S. Pat. No. 5,292,740 (1994) and U.S. Pat. No. 6,136,971 (2000). Two synthetic pathways are known for the preparation of bosentan in the prior art (see Scheme 1).

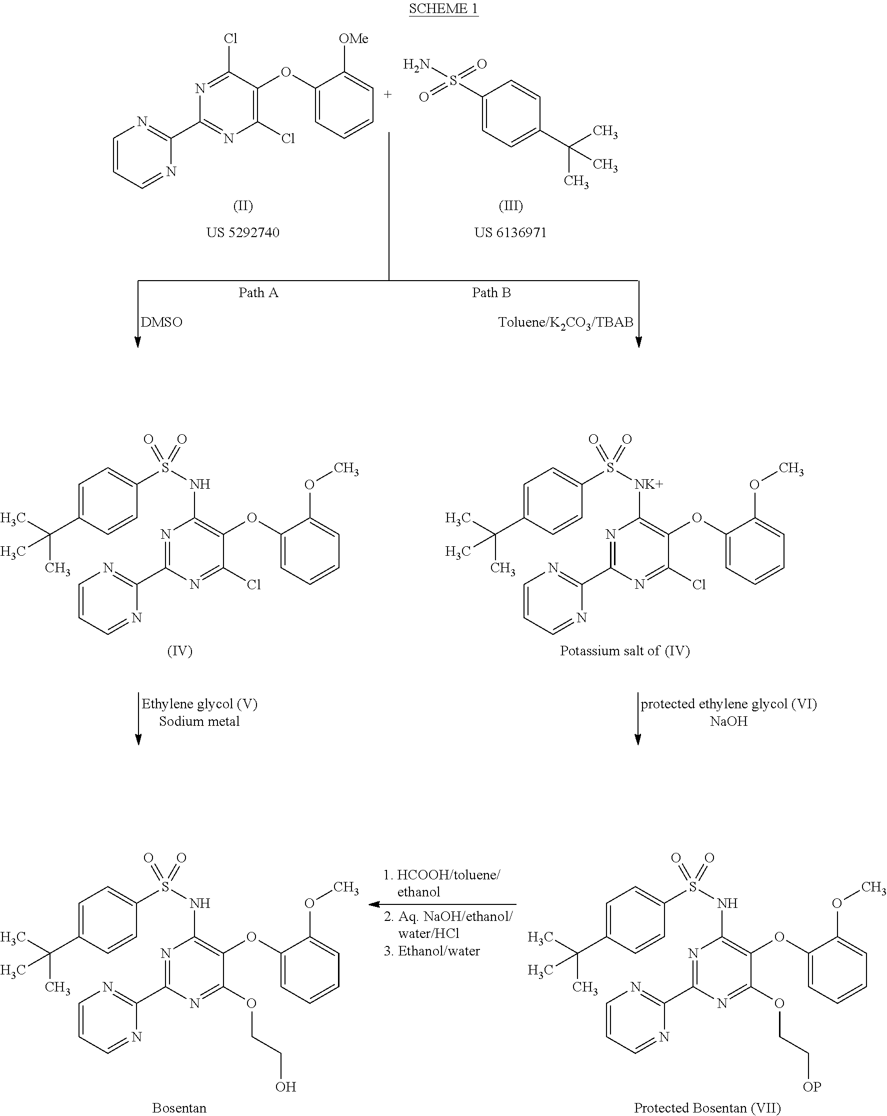

The synthetic path-A described in U.S. Pat. No. 5,292,740 involves the condensation of dichloro pyrimidine (II) with sulfonamide (III) in dimethylsulfoxide (DMSO) to provide p-tert-butyl-N-[6-chloro-5-(2-methoxyphenoxy)-2-(2-pyrimidinyl)-4-pyridinyl]benzenesulfonamide (IV). It was observed that this reaction was incomplete and associated with the formation of several impurities. Hence, isolation of (IV) is difficult without column chromatography.

Subsequently reaction of compound (IV) with sodium ethylene glycolate (prepared by the reaction of ethylene glycol (IV) with sodium metal) yields the sodium salt of bosentan with an overall yield of 50%. The sodium metal used for the preparation of sodium ethylene glycolate is explosive and thus not suitable for industrial preparations. Further, the product formed by this method requires purification by column chromatography in order to control unacceptable amounts of critical impurities A and B (depicted below) and thus to provide pharmaceutical grade bosentan suitable for end use in the drug product.

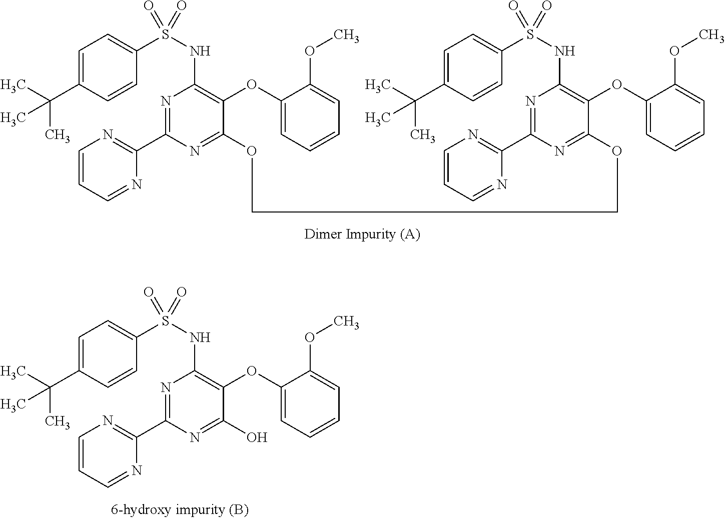

Another synthetic approach (Path-B) described in U.S. Pat. No. 6,136,971 involves the condensation of compound (II) with compound (III) in toluene in presence of anhydrous potassium carbonate and a phase transfer catalyst, benzyl triethylammonium chloride, to provide the potassium salt of (IV). Subsequent reaction of this potassium salt with protected ethylene glycol [ethylene glycol mono-tert-butyl ether (VI)] in toluene in the presence of granular sodium hydroxide yields protected Bosentan (VII). Deprotection of (VII) using formic acid furnished intermediate bosentan formate monoethanolate, which is hydrolysed with sodium hydroxide in absolute ethanol to yielde crude bosentan. The amount of ethylene glycol used in this process is substantially less compared to other processes. The involvement of protection, deprotection and several isolations as purification steps lead to bosentan with too-low yield. Subsequently, a few other processes are reported in WO/2009/095933, WO/2010/032261, WO2009/083739 and WO2009/112954 which synthetic pathway-A in Scheme 1. All the processes of this prior art suffer from the disadvantage of multiple reactions and purification steps, ultimately lowering the yield of bosentan drastically.

As such, there is a need for a high yield process to generate 4-tert-butyl-N-[6-(2-hydroxyethoxy)-5-(2-methoxyphenoxy)-2-(pyrimidin-2-yl)pyrimidin-4-yl]benzene-1-sulfonamide monohydrate (I) (ie. bosentan monohydrate) of high purity which may be directly suitable for pharmaceutical applications and therapeutic use. In particular, there is a need to a minimize the levels of impurities A and B.

### SUMMARY OF THE INVENTION

The present inventors have devised new synthetic processes for preparation of bosentan monohydrate under milder conditions, with shorter reaction times and with fewer steps as compared to known processes. These processes provide bosentan monohydrate at a high level of purity level and at a high yield, thereby rendering the processes of the invention advantageous from an industrial and economical point of view. The processes of the invention in particular provides bosentan monohydrate with low levels of impurities A and B, so that purification by column chromatography is not require.

Accordingly, the present invention provides a process for the preparation of bosentan monohydrate (I):

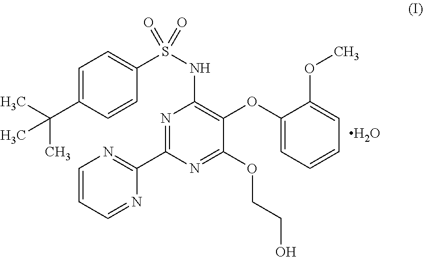

which process comprises:

(a) condensation of 4-tert-butyl-N-[6-chloro-5-(2-methoxyphenoxy)-2-(2-pyrimidinyl)-4-pyridinyl]benzene sulfonamide potassium salt (potassium salt of (IV)):

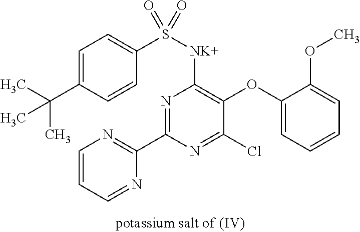

with ethylene glycol (V) in the presence of an inorganic base to produce bosentan potassium salt (VI);

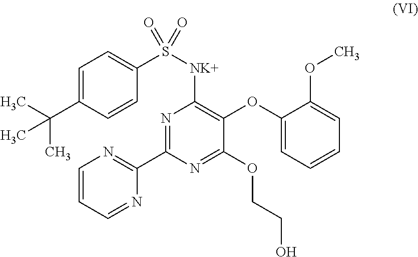

(b) isolation of bosentan by quenching of the reaction mixture from step (a) followed by acidification and filtration;

(c) purification of bosentan from step (b) formation of bosentan potassium salt;

(d) recrystallization of bosentan potassium salt from step (c) using a mixed solvent system;

(e) formation of bosentan by acidification of bosentan potassium salt from step (d);

(f) recrystallization of bosentan from step (e) using a polar mixed solvent system; and

(g) hydration of bosentan from step (f) to provide bosentan monohydrate.

The invention further provides a process for producing 4-tert-butyl-N-[6-chloro-5-(2-methoxyphenoxy)-2-(2-pyrimidinyl)-4-pyridinyl]benzene sulfonamide potassium salt (potassium salt of (IV)), which process comprises:

(h) condensation of 4,6 dichloro-5-(2-methoxybenzyl)-2,2-bipyrimidine (II) with 4-tert-butylbenzenesulphonamide (III) in the presence of an inorganic base and an ethereal solvent; and

(i) optionally quenching the reaction mixture, preferably in water, and optionally isolating 4-tert-butyl-N-[6-chloro-5-(2-methoxyphenoxy)-2-(2-pyrimidinyl)-4-pyridinyl]benzene sulfonamide potassium salt by filtration.

The invention further provides a process for the preparation of bosentan monohydrate (I) comprising:

(j) condensation of 4,6 dichloro-5-(2-methoxybenzyl)-2,2-bipyrimidine (II) with 4-tert-butylbenzenesulphonamide (III) in the presence of an inorganic base;

(k) in situ condensation of thus formed 4-tert-butyl-N-[6-chloro-5-(2-methoxyphenoxy)-2-(2-pyrimidinyl)-4-pyridinyl]benzene sulfonamide potassium salt (potassium salt of (IV)) with ethylene glycol (V);

(l) quenching of reaction mixture from step (k) and isolation of bosentan potassium salt(VI);

(m) recrystallization of bosentan potassium salt from step (l) using a mixed solvent system;

(n) formation of bosentan by acidification of bosentan potassium salt from step (m);

(o) recrystallization of bosentan from step (n) using a polar mixed solvent system; and

(p) hydration of bosentan from step (o) to provide bosentan monohydrate.

### DETAILED DESCRIPTION OF THE INVENTION

Two examples of overall processes of the current invention are depicted in Scheme 2 below.

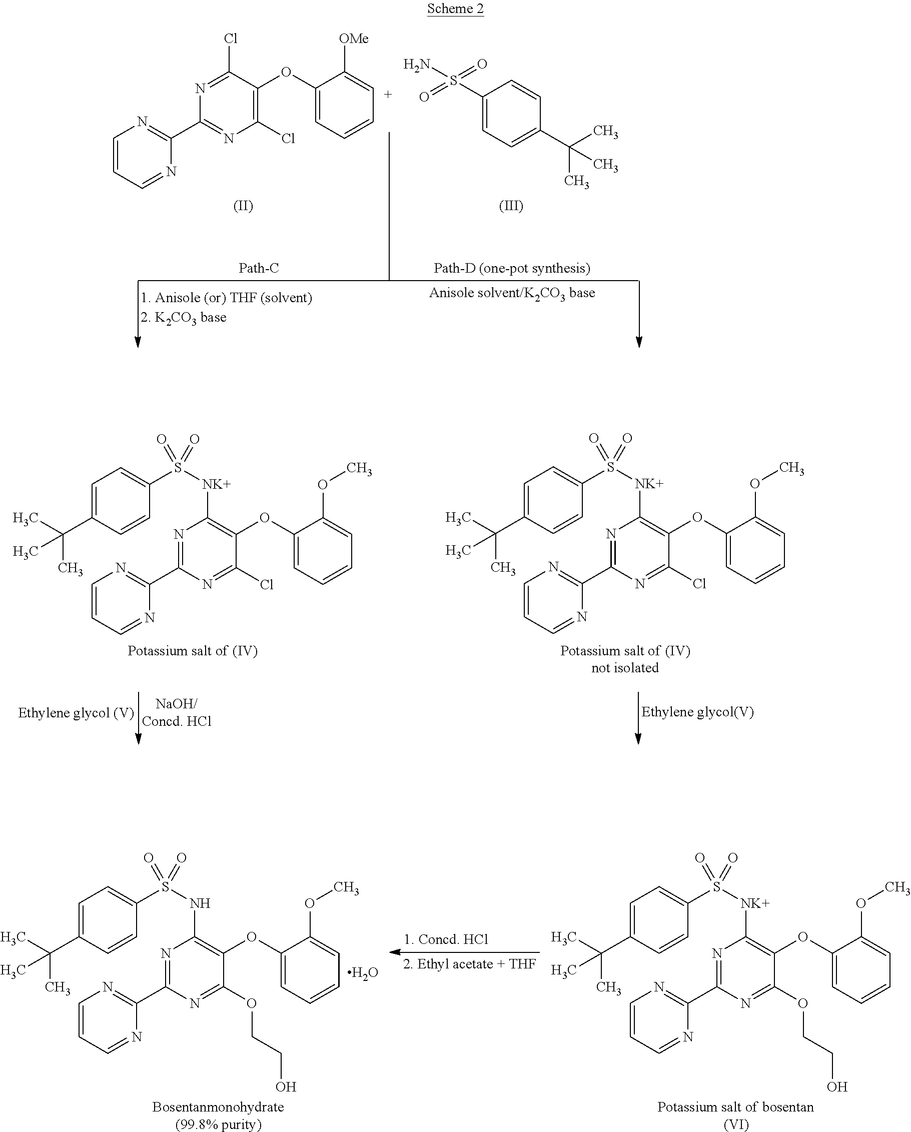

The synthetic path-C of the current invention initially involves in step (h) the condensation of 4,6 dichloro-5-(2-methoxybenzyl)-2,2-bipyrimidine (II) with 4-tert-butylbenzenesulphonamide (III) in the presence of an inorganic base and an ethereal solvent.

Typically, the inorganic base is potassium carbonate in step (h). Preferably, step (h) is carried out using between about 1.0 and about 3.0 molar equivalents of potassium carbonate base and particularly about 2.0 molar equivalents of potassium carbonate with respect to 4,6 dichloro-5-(2-methoxybenzyl)-2,2-bipyrimidine (II).

An ethereal solvent is a solvent containing an ether moiety. Preferred ethereal solvents are anisole and THF. Preferably, the reaction is carried out in THF or anisole at 65-150° C.

After completion of the reaction in step (h), the reaction mixture is typically quenched, preferably in water. It is then typically filtered and washed with a solvent, such as THF, to yield the potassium salt of (IV). The yield of the potassium salt of (IV) is typically greater than 90%, for example about 96%. Purity is typically greater than 95%, preferably greater than 99%, for example about 99.7%.

The potassium salt of (IV) is then condensed in step (a) with ethylene glycol (V) in the presence of an inorganic base.

Typically, step (a) is carried out using between 60.0 and 125.0 molar equivalents, and particularly about 100.0 molar equivalents, of ethylene glycol (V) with respect to potassium salt of (IV).

Typically, the solvent in step (a) is anisole or acetonitrile.

Typically, the inorganic base in step (a) is sodium hydroxide or potassium carbonate.

When the inorganic base is sodium hydroxide, the amount of sodium hydroxide used is preferably between about 3.0 and 5.0 molar equivalents, and more preferably about 4.0 molar equivalents, with respect to potassium salt of (IV). Typically, the reaction of step (a) is carried out at a temperature of 80-100° C. and preferably 90-95° C. when the base is sodium hydroxide.

When the inorganic base is potassium carbonate, the amount of potassium carbonate used is preferably between 5.0 and 9.0 molar equivalents, and more preferably about 6.0 molar equivalents with respect to potassium salt of (IV). Typically, the reaction is carried out at a temperature range of 70-90° C. and preferably about 80° C. when the base is potassium carbonate.

In step (b), the reaction mixture from step (a) is quenched, then acidified and filtered, to isolate bosentan. Quenching in step (b) is typically achieved by pouring the reaction mixture from step (a) into water or a mixed solvent system, which is preferably a mixture of acetonitrile and water. Acidification is typically achieved by addition of hydrochloric acid, preferably concentrated hydrochloric acid.

The yield of bosentan in step (b) is typically greater than 80%, for example about 83% or about 85%. Purity is typically greater than 95%, preferably greater than 97%, for example about 97.5%. The bosentan from step (b) typically contains 1 to 2% of impurity B and 0.3 to 0.6% of impurity A.

In step (c), bosentan from step (b) is purified by formation of the potassium salt of bosentan. This purification step reduces the levels of impurity A. Typically, the potassium salt of bosentan is formed by reaction with potassium hydroxide, preferably in a mixed solvent system. Preferably, a solvent system comprising acetonitrile and water is used in step (c). More preferably the solvent system is aqueous acetonitrile, wherein the volume ratio of acetonitrile to water is 80:20 to 99:1, preferably 90:10 to 98:2, for example about 95:5. Typically, step (c) is performed at reflux temperature.

In step (d), the bosentan potassium salt from step (c) is recrystallized using a mixed solvent system. This purification step reduces the levels of impurity B. Typically, a solvent system comprising acetonitrile and water is used to recrystallize the bosentan potassium salt. Preferably, the solvent system in step (d) is aqueous acetonitrile, wherein the volume ratio of acetonitrile to water is 1:99 to 20:80 to, preferably 2:98 to 10:90, for example about 5:95.

In step (e), bosentan is formed by acidification of bosentan potassium salt from step (d). Acidification is typically achieved by addition of hydrochloric acid, preferably concentrated, hydrochloric acid. Bosentan is then typically extracted, preferably using ethylacetate as the solvent.

In step (f), bosentan from step (e) is recrystallized using a polar mixed solvent system. Typically, a solvent system comprising one or more polar solvents is used in step (f). Preferably, a solvent system comprising ethyl acetate and THF or ethyl acetate and acetone is used in step (f). More preferably a solvent system comprising ethyl acetate and THF is used.

In step (g), bosentan monohydrate is formed by hydration of bosentan from step (f). Typically, hydration involves precipitation of bosentan monohydrate from bosentan using a solvent system comprising methanol and water.

The bosentan monohydrate from step (g) is typically a white crystalline powder. The purity is typically greater than 99%, preferably greater than 99.5%, for example about 99.8%. Typically the bosentan monohydrate from step (g) contains less than 0.1%, for example about 0.03%, of impurity B and less than 0.1%, for example about 0.08% of impurity A.

The synthetic path-D of the current invention initially involves in step (j) the condensation of 4,6 dichloro-5-(2-methoxybenzyl)-2,2-bipyrimidine (II) with 4-tert-butylbenzenesulphonamide (III) in the presence of an inorganic base.Typically, the inorganic base in step (j) is potassium carbonate. Preferably, the condensation of step (j) is carried out using between about 5.0 and 9.0 molar equivalents of potassium carbonate base and particularly about 6.0 molar equivalents of potassium carbonate with respect to 4,6 dichloro-5-(2-methoxybenzyl)-2,2-bipyrimidine (II).

Typically, the solvent in step (j) is anisole. Preferably, step (j) is carried out in anisole solvent at a temperature of 110 to 130° C., more preferably 120-125° C., for example about 120° C. The reaction time for step (j) is typically 5 to 8 hours, preferably 6 to 7 hours.

The condensation of step (j) is typically monitored, for example by thin layer chromatography (TLC), for the absence of 4,6 dichloro-5-(2-methoxybenzyl)-2,2-bipyrimidine (II). Generally, once 4,6 dichloro-5-(2-methoxybenzyl)-2,2-bipyrimidine is absent or substantially absent (such that it cannot be detected), ethylene glycol is charged in step (k).

Ethylene glycol is charged in step (k) in situ, that is to say without prior isolation or separation of the bosentan potassium salt (VI) formed in step (j). This is thus a “one-pot” synthesis. By reducing the number of isolation and separation steps, the yield of the process is increased.

Typically, the amount of ethylene glycol used in step (k) is between 100.0 and 125.0 molar equivalents, and particularly about 125.0 molar equivalents, with respect to (II).

Typically, the reaction mixture from step (j) is at a temperature of 110 to 130° C., for example about 120° C., when the ethylene glycol is added. Typically, the ethylene glycol is at a temperature of 110 to 130° C., for example about 120° C., when it is added to the reaction mixture from step (j). Preferably, therefore, both the ethylene glycol and the reaction mixture from step (j) are at a temperature of 110 to 130° C., for example about 120° C., when they are mixed.

Typically, the condensation of step (k) is carried out at a temperature of 110 to 130° C., for example about 120° C. Typically the reaction time for step (k) is 2 to 5 hours, preferably 3 to 4 hours.

The reaction mixture from step (k) is then quenched in step (l) and bosentan potassium salt (VI) is isolated. Typically, quenching is achieved in step (l) by pouring the reaction mixture from step (k) into an excess of water. Typically, isolation of bosentan potassium salt is achieved by filtration.

The bosentan potassium salt isolated in step (k) is typically more than 97% pure, for example about 98% pure. Typically, about 0.2% of impurity-B and about 0.15% impurity-A are present.

In step (m), the bosentan potassium salt from step (l) is recrystallized using a mixed solvent system. Typically, a solvent system comprising acetonitrile and water is used to recrystallize the bosentan potassium salt. Preferably the solvent system in step (l) is aqueous acetonitrile, wherein the volume ratio of acetonitrile to water is 1:99 to 20:80 to, preferably 2:98 to 10:90, for example about 5:95.

The bosentan potassium salt from step (m) is then converted into bosentan by acidification in step (n). Acidification is typically achieved by addition of hydrochloric acid, preferably concentrated hydrochloric acid. Bosentan is then typically isolated, for example by filtration.

Bosentan from step (n) is then recrystallized in step (o) using a polar mixed solvent system. Typically, a solvent system comprising one or more polar solvents is used in step (o). Preferably, a solvent system comprising ethyl acetate and acetone or ethylacetate and THF is used in step (o). A mixture of ethyl acetate and acetone is preferred.

In step (p), bosentan monohydrate is formed by hydration of bosentan from step (o). Typically, hydration involves precipitation of bosentan monohydrate using a solvent system comprising methanol and water.

The bosentan monohydrate from step (p) is typically a pure white crystalline powder. The purity is typically greater than 99%, preferably greater than 99.5%, for example about 99.9%. Typically the bosentan monohydrate from step (p) contains less than 0.1%, for example about 0.01%, of impurity B and less than 0.1%, for example 0.02% of impurity A.

The total yield of bosentan monohydrate obtained by the processes of the invention, via either path C or path D, is typically greater than 30%, for example about 40%. The purity of the bosentan prepared by the processes of the invention is typically greater than 98%, preferably greater than 99%, more preferably greater than 99.5%, for example 99.8% or 99.9%, as compared to 90 to 95% from prior art processes.

The levels of impurities A and B are also low in the bosentan monohydrate obtained from the processes of the invention:

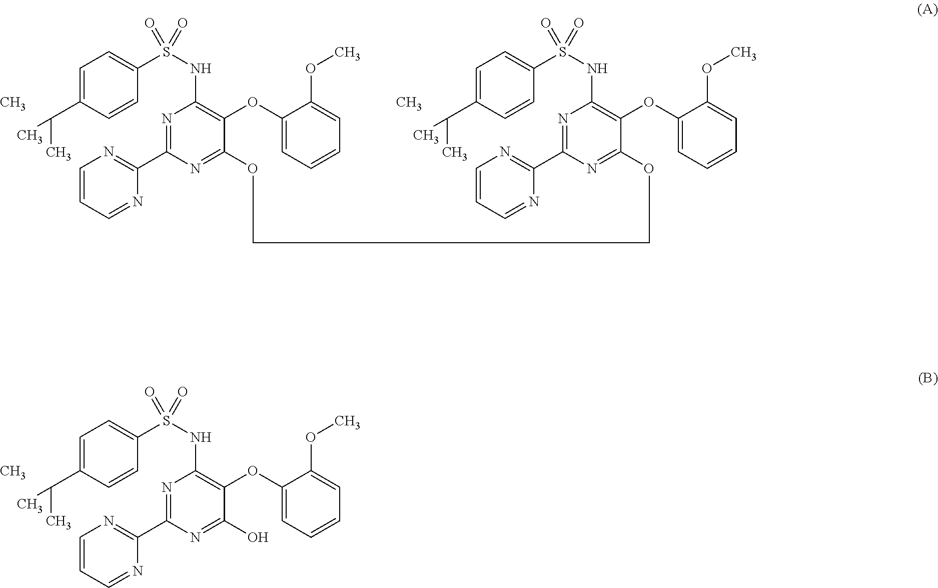

Typically, the levels of impurity A are less than 0.1%, preferably less than 0.05%. Typically, the levels of impurity B are less than 0.1%, preferably less than 0.05%. Preferably the total levels of impurities A and B are less than 0.2%, such that the bosentan monohydrate is 99.8% pure or greater. In consequence, the processes of the invention do not require use of column chromatography to purify the product; such techniques are expensive and time consuming, and reduce the overall yield of the product.

Purity of products can be measured using any suitable technique known to those skilled in the art. High-pressure liquid chromatography (HPLC) is a preferred technique. Purity is typically measure as percentage purity by weight.

The following Examples are provided to illustrate the invention. The Examples are not meant to limit the scope of the invention as defined in the claims.

### EXAMPLES

### Example 1

### Preparation of 4-tert-butyl-N-[6-(2-hydroxyethoxy)-5-(2-methoxyphenoxy)-2-(pyrimidin-2-yl)pyrimidin-4-yl]benzene-1-sulfonamide monohydrate (I)

### Step-A: 4-tert-butyl-N-[6-chloro-5-(2-methoxyphenoxy)-2-(2-pyrimidinyl)-4-pyridinyl]benzene sulfonamide potassium salt (potassium salt of (IV))

4,6 dichloro-5-(2-methoxybenzyl)-2,2-bipyrimidine (II) (100 g, 0.286 moles) and anisole (1250 ml) were placed in a reaction flask. Potassium carbonate (79 g, 0.572 moles) was added and reaction mass was stirred for 15 minutes. 4-tert-butylbenzenesulphonamide (III) (213 g, 0.286 moles) was added to reaction mass and heated to 140° C. for three hours. After completion of reaction the reaction mass was brought to room temperature and poured into purified water. The solid product was filtered off, washed with THF and dried to afford 4-tert-butyl-N-[6-chloro-5-(2-methoxyphenoxy)-2-(2-pyrimidinyl)-4-pyridinyl]benzene sulfonamide potassium salt.

(161 g, yield 96.2% , purity by HPLC: 99.7%)

### Step-B: Preparation of bosentan

Ethylene glycol (550.6 g, 8.88 moles) and sodium hydroxide (14.9 g, 0.376 moles) were placed in reaction flask and stirred for 15 minutes. Reaction mass temperature was raised to 95° C. and 4-tert-butyl-N-[6-chloro-5-(2-methoxyphenoxy)-2-(2-pyrimidinyl)-4-pyridinyl]benzene sulfonamide potassium salt (50 g, 0.088 moles) obtained from step-A was charged. Reaction mass was maintained at 90-95° C. for 3 hours, brought to room temperature and poured into a mixture of acetonitrile and water and acidified with concentrated hydrochloric acid. The precipitated solid was filtered and dried to afford bosentan.

(42 g, yield 83.1%, purity by HPLC: 97.6% with 0.43% impurity-A and 1.7% impurity-B)

### Step-C: Purification of bosentan

i. Bosentan from step-B (41 g, 0.0720 moles) and acetonitrile (390 ml) were placed in a reaction flask. Potassium hydroxide (6 g, 0.108 moles) dissolved in purified water (20.5 ml) was added to reaction mass and heated to 60° C. for 30 minutes. The reaction mass was brought to room temperature and stirred for 2 hours. The solid product was filtered off, washed with acetonitrile to afford bosentan potassium salt (48 g, purity by HPLC: 98.8% with 0.169% impurity-A and 0.77% impurity-B).
ii. The bosentan potassium salt obtained above was recrystallized twice from aqueous acetonitrile (5% acetonitrile, 206 ml) to yield bosentan potassium salt (38 g, purity by HPLC: 99.75% purity with 0.157% impurity-A and 0.028% impurity-B).

### Step-D: Preparation of bosentan monohydrate

The bosentan potassium salt obtained from step-C was suspended in purified water and acidified with concentrated hydrochloric acid. Liberated bosentan was filtered and recrystallized with a mixture of ethyl acetate and THF and obtained bosentan was precipitated from a mixture of methanol and water to yield bosentan monohydrate as pure white crystalline powder. (25.6 g, purity by HPLC: 99.85% with 0.08% impurity-A and 0.006% impurity-B)

### Example-2

### Preparation of 4-tert-butyl-N-[6-(2-hydroxyethoxy)-5-(2-methoxyphenoxy)-2-(pyrimidin-2-yl)pyrimidin-4-yl]benzene-1-sulfonamide monohydrate (I)

### Step-A: 4-tert-butyl-N-[6-chloro-5-(2-methoxyphenoxy)-2-(2-pyrimidinyl)-4-pyridinyl]benzene sulfonamide potassium salt (potassium salt of (IV))

4,6 dichloro-5-(2-methoxybenzyl)-2,2-bipyrimidine (II) (100 g, 0.286 moles) and THF (1000 ml) were placed in a reaction flask. Potassium carbonate (79 g, 0.572 moles) was added and reaction mass was stirred for 15 minutes. 4-tert-butylbenzenesulphonamide (III) (213 g, 0.286 moles) was added to reaction mass and heated to 75° C. for 20 hours. After reaction completion reaction mass was brought to room temperature and poured into purified water. The solid product was filtered off, washed with THF and dried to afford 4-tert-butyl-N-[6-chloro-5-(2-methoxyphenoxy)-2-(2-pyrimidinyl)-4-pyridinyl]benzene sulfonamide potassium salt.

(147.2 g, yield 88% , purity by HPLC: 99.6%)

### Step-B: Preparation of bosentan

Ethylene glycol (550.6 g, 8.88 moles), acetonitrile (500 ml) and potassium carbonate (98 g, 0.710 moles) were placed in reaction flask and stirred for 15 minutes. Reaction mass temperature was raised to 55° C. and charged 4-tert-butyl-N-[6-chloro-5-(2-methoxyphenoxy)-2-(2-pyrimidinyl)-4-pyridinyl]benzene sulfonamide potassium salt (50 g, 0.088 moles) obtained from step-A. Reaction mass was refluxed at 80-85° C. for 12 hours and brought to room temperature. Reaction mass was poured into purified water and acidified with concentrated hydrochloric acid. The precipitated solid was isolated by filtration and dried to afford bosentan.

(45 g, yield 89%, purity by HPLC: 97.8% with 0.6% impurity-A and 1% impurity-B)

### Step-C: Purification of bosentan

i. Bosentan from step-B (44 g, 0.0773 moles) and acetonitrile (390 ml) were placed in a reaction flask. Potassium hydroxide (6.4 g, 0.115 moles) dissolved in purified water (22 ml) was added to reaction mass and heated to 60° C. for 30 minutes. Reaction mass was brought to room temperature and stirred for 2 hours. The solid product was filtered off, washed with acetonitrile to afford bosentan potassium salt (51 g, purity by HPLC: 98.8% with 0.24% impurity-A and 0.41% impurity-B).
ii. The bosentan potassium salt obtained above was recrystallized from aqueous acetonitrile (5% acetonitrile, 206 ml) to yield bosentan potassium salt (41 g, purity by HPLC: 99.5% purity with 0.185% impurity-A and 0.015% impurity-B).

### Step-D: Preparation of bosentan monohydrate

The bosentan potassium salt obtained from step-C was suspended in ethyl acetate and purified water mixture and acidified with concentrated hydrochloric acid. Ethyl acetate layer was washed with brine solution, distilled off completely and recrystallized with a mixture of ethyl acetate and THF and obtained bosentan was precipitated from a mixture of methanol and water to yield bosentan monohydrate as pure white crystalline powder.

(27.6 g, purity by HPLC: 99.8% with 0.09% impurity-A and 0.005% impurity-B)

### Example-3

### Preparation of 4-tert-butyl-N-[6-(2-hydroxyethoxy)-5-(2-methoxyphenoxy)-2-(pyrimidin-2-yl)pyrimidin-4-yl]benzene-1-sulfonamide monohydrate(I)

### Step-A: Preparation of 4-tert-butyl-N-[6-(2-hydroxyethoxy)-5-(2-methoxyphenoxy)-2-(pyrimidin-2-yl)pyrimidin-4-yl]benzene-1-sulfonamide potassium salt (bosentan potassium salt (VI))

4,6 dichloro-5-(2-methoxybenzyl)-2,2-bipyrimidine (II) (50 g, 0.143 moles) and anisole (750 ml) were placed in a reaction flask. Potassium carbonate (169.9 g, 1.23 moles) was added and reaction mass was stirred for 15 minutes. 4-tert-butylbenzenesulphonamide (III) (30.5 g, 0.143 moles) was added to reaction mass and heated to 120° C. for 6 hours. Ethylene glycol (550.6 g, 8.88 moles) was charged to reaction mass and maintained at 120° C. for 3 hours and brought to room temperature. Reaction mass was poured into purified water (3 L) and stirred for 30 minutes. The precipitated solid was isolated by filtration and washing with acetonitrile to afford bosentan potassium salt.

(81 g, yield 89%, purity by HPLC: 87.4% with 0.42% impurity-A , 0.7% impurity-B and 11% 4-tert-butylbenzenesulphonamide (III))

### Step-B: Purification of bosentan potassium salt

The bosentan potassium salt obtained above was recrystallized from aqueous acetonitrile (5% water, 810 ml) to yield bosentan potassium salt.

(54.0 g, purity by HPLC: 99.67% purity with 0.08% impurity-A ; 0.159% impurity-B and 4-tert-butylbenzenesulphonamide (III)—not detected)

### Step-C: Preparation of bosentan monohydrate

The bosentan potassium salt obtained from step-B was suspended in purified water and acidified with concentrated hydrochloric acid. Liberated bosentan was recrystallized with a mixture of ethyl acetate and THF to yield bosentan and obtained bosentan was precipitated from a mixture of methanol and water to yield bosentan monohydrate as pure white crystalline powder.

(30 g, purity by HPLC: 99.85% (with 0.06% impurity-A and 0.04% impurity-B)

### Example-4

### Preparation of 4-tert-butyl-N-[6-(2-hydroxyethoxy)-5-(2-methoxyphenoxy)-2-(pyrimidin-2-yl)pyrimidin-4-yl]benzene-1-sulfonamide monohydrate(I)

### Step-A: Preparation of 4-tert-butyl-N-[6-(2-hydroxyethoxy)-5-(2-methoxyphenoxy)-2-(pyrimidin-2-yl)pyrimidin-4-yl]benzene-1-sulfonamide potassium salt (bosentan potassium salt (VI))

4,6 dichloro-5-(2-methoxybenzyl)-2,2-bipyrimidine (II) (50 g, 0.143 moles) and anisole (750 ml) were placed in a reaction flask. Potassium carbonate (169.9 g, 1.23 moles) was added and reaction mass was stirred for 15 minutes. 4-tert-butylbenzenesulphonamide (III) (30.5 g, 0.143 moles) was added to reaction mass and heated to 120° C. for 6 hours. Ethylene glycol (888 g, 14.3 moles) was charged to reaction mass and maintained at 120° C. for 3 hours and brought to room temperature. Reaction mass was poured into purified water (3 L) and stirred for 30 minutes. The precipitated solid was isolated by filtration, washing with acetonitrile and drying to afford bosentan potassium salt.

(79 g, yield 89.7%, purity by HPLC: 84.3% with 0.36% impurity-A , 1.1% impurity-B and 14% 4-tert-butylbenzenesulphonamide (III))

### Step-B: Purification of bosentan potassium salt

The bosentan potassium salt obtained above was recrystallized from aqueous acetonitrile (5% water, 810 ml) to yield bosentan potassium salt.

(53.0 g, purity by HPLC: 99.44% purity with 0.116% impurity-A ; 0.411% impurity-B and 4-tert-butylbenzenesulphonamide (III)—not detected)

### Step-C: Preparation of bosentan monohydrate

The bosentan potassium salt obtained from step-B was suspended purified water and acidified with concentrated hydrochloric acid. Liberated bosentan was recrystallized with a mixture of ethyl acetate and THF to yield bosentan monohydrate and obtained bosentan was precipitated from a mixture of methanol and water to yield bosentan monohydrate as pure white crystalline powder.

(32 g, purity by HPLC: 99.85% (with 0.07% impurity-A and 0.04% impurity-B)

### Example-5

### Preparation of 4-tert-butyl-N-[6-(2-hydroxyethoxy)-5-(2-methoxyphenoxy)-2-(pyrimidin-2-yl)pyrimidin-4-yl]benzene-1-sulfonamide monohydrate(I)

### Step-A: Preparation of 4-tert-butyl-N-[6-(2-hydroxyethoxy)-5-(2-methoxyphenoxy)-2-(pyrimidin-2-yl)pyrimidin-4-yl]benzene-1-sulfonamide potassium salt (bosentan potassium salt (VI))

4,6 dichloro-5-(2-methoxybenzyl)-2,2-bipyrimidine (II) (50 g, 0.143 moles) and anisole (750 ml) were placed in a reaction flask. Potassium carbonate (118.4 g, 0.858 moles) was added and reaction mass was stirred for 15 minutes. 4-tert-butylbenzenesulphonamide (III) (30.5 g, 0.143 moles) was added to reaction mass and heated to 120° C. for 7 hours. Ethylene glycol (1.110 g, 17.9 moles) was charged to reaction mass and maintained at 120° C. for 5 hours and brought to room temperature. Reaction mass was poured into purified water (4.5 L) and stirred for 30 minutes. The precipitated solid was isolated by filtration and washing with acetonitrile to afford bosentan potassium salt.

(83 g, yield 90%, purity by HPLC: 91.2% with 0.159% impurity-A, 0.686% impurity-B and 7.8% 4-tert-butylbenzenesulphonamide (III))

### Step-B: Purification of bosentan potassium salt

The bosentan potassium salt obtained above was recrystallized from aqueous acetonitrile (5% water, 810 ml) to yield bosentan potassium salt.

(50.5 g, purity by HPLC: 99.00% purity with 0.048% impurity-A; 0.123% impurity-B and 4-tert-butylbenzenesulphonamide (III)—0.78)

### Step-C: Preparation of bosentan monohydrate

The bosentan potassium salt obtained from step-B was suspended in purified water and acidified with concentrated hydrochloric acid. Liberated bosentan was filtered, recrystallized with a mixture of ethyl acetate and acetone. Obtained bosentan was precipitated from a mixture of methanol and water to yield bosentan monohydrate as pure white crystalline powder.

(30 g, purity by HPLC: 99.90% (with 0.02% impurity-A and 0.01% impurity-B))

1. A process for the preparation of bosentan monohydrate (I): 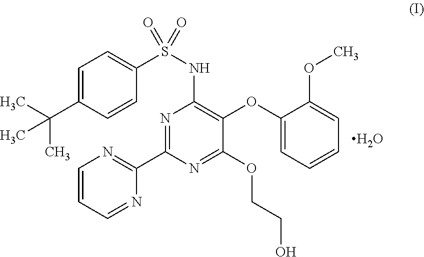
which process comprises:
(a) condensation of 4-tert-butyl-N-[6-chloro-5-(2-methoxyphenoxy)-2-(2-pyrimidinyl)-4-pyridinyl]benzene sulfonamide potassium salt (potassium salt of (IV)): 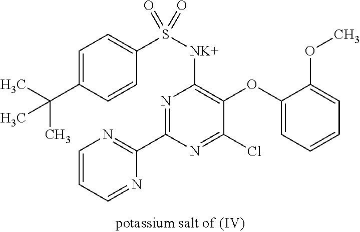
with ethylene glycol (V) in the presence of an inorganic base to produce bosentan potassium salt (VI); 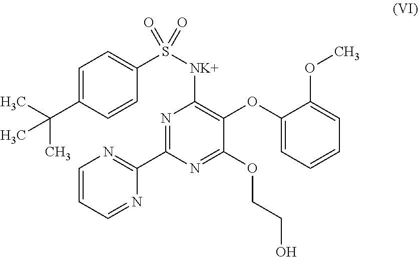
(b) isolation of bosentan by quenching of the reaction mixture from step (a) followed by acidification and filtration;
(c) purification of bosentan from step (b) by formation of bosentan potassium salt;
(d) recrystallization of bosentan potassium salt from step (c) in a mixed solvent system;
(e) formation of bosentan by acidification of bosentan potassium salt from step (d);
(f) recrystallization of bosentan from step (e) in a polar mixed solvent system; and
(g) hydration of bosentan from step (f) to provide bosentan monohydrate.

2. A process according to claim 1, wherein the condensation of step (a) uses anisole or acetonitrile as a solvent.

3. A process according to claim 1, wherein the inorganic base in step (a) is sodium hydroxide or potassium carbonate.

4. A process according to claim 1, wherein bosentan is isolated in step (b) by quenching the reaction mixture from step (a) in water or mixture of acetonitrile and water.

5. A process according to claim 1, wherein the bosentan potassium salt is formed in step (c) by reaction with potassium hydroxide in a mixed solvent system which preferably comprises acetonitrile and water.

6. A process according to claim 1, wherein a solvent system comprising acetonitrile and water is used is used to recrystallize the bosentan potassium salt in step (d).

7. A process according to claim 1, wherein a solvent system comprising ethyl acetate and THF is used to recrystallize the bosentan in step (f).

8. A process according to claim 1, wherein bosentan monohydrate is formed in step (g) by addition of bosentan from step (f) to a solvent system comprising methanol and water.

9. A process for producing 4-tert-butyl-N-[6-chloro-5-(2-methoxyphenoxy)-2-(2-pyrimidinyl)-4-pyridinyl]benzene sulfonamide potassium salt (potassium salt of (IV)), which process comprises:
(h) condensation of 4,6 dichloro-5-(2-methoxybenzyl)-2,2-bipyrimidine (II) with 4-tert-butylbenzenesulphonamide (III) in the presence of an inorganic base and anisole; and
(i) optionally quenching the reaction mixture, preferably in water, and optionally isolating 4-tert-butyl-N-[6-chloro-5-(2-methoxyphenoxy)-2-(2-pyrimidinyl)-4-pyridinyl]benzene sulfonamide potassium salt by filtration.

10. (canceled)

11. A process according to claim 9, where in the inorganic base is potassium carbonate.

12. A process for the preparation of bosentan monohydrate (I) comprising:
(j) condensation of 4,6 dichloro-5-(2-methoxybenzyl)-2,2-bipyrimidine (II) with 4-tert-butylbenzenesulphonamide (III) in the presence of an inorganic base and anisole;
(k) in situ condensation of thus formed 4-tert-butyl-N-[6-chloro-5-(2-methoxyphenoxy)-2-(2-pyrimidinyl)-4-pyridinyl]benzene sulfonamide potassium salt (potassium salt of (IV)) with ethylene glycol (V);
(l) quenching of reaction mixture from step (k) and isolation of bosentan potassium salt (VI);
(m) recrystallization of bosentan potassium salt from step (l) using a mixed solvent system;
(n) formation of bosentan by acidification of bosentan potassium salt from step (m);
(o) recrystallization of bosentan from step (n) using a polar mixed solvent system; and
(p) hydration of bosentan from step (o) to provide bosentan monohydrate.

13. (canceled)

14. A process according to claim 12, where in the inorganic base in step (j) is potassium carbonate.

15. A process according to claim 12, wherein the condensation of step (j) is carried out at a temperature of 110 to 130° C.

16. A process according to claim 12, wherein the reaction mixture from step (j) is a temperature of 110 to 130° C. when ethylene glycol is added in step (k).

17. A process according to claim 12, wherein the condensation of step (k) is carried out at a temperature of 110 to 130° C.

18. A process according to claim 12, wherein bosentan potassium salt (VI) is isolated in step (l) by quenching the reaction mixture from step (k) in water.

19. A process according to claim 12, wherein a solvent system comprising acetonitrile and water is used to recrystallize the bosentan potassium salt in step (m).

20. A process according to claim 12, wherein a solvent system comprising ethyl acetate and acetone is used to the recrystallize the bosentan in step (o).

21. A process according to claim 12, wherein bosentan monohydrate is formed in step (p) by addition of bosentan from step (o) to a solvent system comprising methanol and water.

22. A process according to claim 12, wherein the overall yield of bosentan monohydrate is greater than 35%.

23. A process according to claim 12, wherein the purity of the resulting bosentan monohydrate is greater than 98%, preferably greater than 99.5%, more preferably greater than 99.8%.

24. A process according to claim 12, wherein less than 0.1%. of compound (A) is present in the resulting bosentan monohydrate.

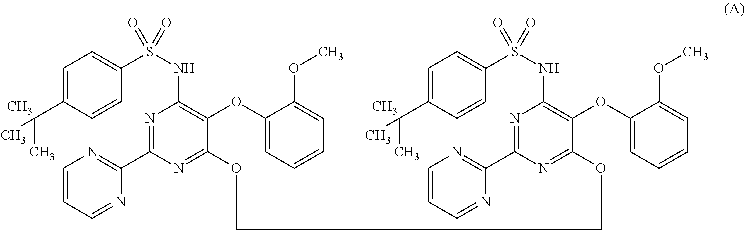

25. A process according to claim 12, wherein less than 0.1% of compound (B) is present in resulting bosentan monohydrate. [chemical structure]
